(19) United States
(12) Patent Application Publication
(10) Pub. No.: US 2026/0027024 A1
(43) Pub. Date: Jan. 29, 2026

(54) ANTIBACTERIAL TOOTHPASTE

(71) Applicants: PEKING UNIVERSITY SCHOOL OF STOMATOLOGY, Beijing (CN); BEIJING PIEZO-DENT MEDICAL TECHNOLOGY CO., LTD, Beijing (CN)

(72) Inventors: Xuehui ZHANG, Beijing City (CN); Xuliang DENG, Beijing City (CN); Mingming XU, Beijing City (CN); Yanhui LU, Beijing City (CN)

(21) Appl. No.: 19/138,557

(22) PCT Filed: Dec. 12, 2023
(86) PCT No.: PCT/CN2023/138006

§ 371 Date: Jun. 12, 2025

(30) Foreign Application Priority Data
Dec. 16, 2022 (CN) 202211618989.3

(51) Int. Cl. A61K 8/29; A61K 8/02; A61Q 11/00

(52) U.S. Cl. CPC A61K 8/29; A61K 8/0245; A61K 8/027; A61Q 11/00; A61K 2800/28; A61K 2800/412; A61K 2800/61; A61K 2800/81

(57) ABSTRACT

The invention provides an antibacterial toothpaste, comprising a charged antibacterial toothpaste abrasive. The charged antibacterial toothpaste abrasive comprises a ferroelectric ceramic. The ferroelectric ceramic is selected from at least one or a compound or a composition of more than one of potassium sodium niobate, i.e. K_{0.5}Na_{0.5}NbO₃, barium titanate, i.e. BaTiO₃, lithium niobate, i.e. LiNbO₃, and barium strontium titanate, i.e. BaₓSr₁₋ₓTiO₃, where 0<x<1. In the inventive antibacterial toothpaste, the charged antibacterial toothpaste abrasive can maintain good electrical stability, such that an antibacterial toothpaste containing same can play a lasting antibacterial role by utilizing a polarization effect, and has a relatively good antibacterial performance, thereby achieving both teeth cleaning and antibacterial effects.

### CROSS-REFERENCE TO RELATED APPLICATION

The present application claims priority to application No. CN202211618989.3 entitled “ANTIBACTERIAL TOOTHPASTE” filed on Dec. 16, 2022, the disclosure of which is incorporated herein by reference in its entirety.

### TECHNICAL FIELD

The present application relates to the technical field of toothpaste, and in particular to an antibacterial toothpaste.

### BACKGROUND

The abrasive is the most abundant ingredient in toothpaste, and mainly plays a role in cleaning teeth. At present, toothpaste abrasives are mostly composed of calcium carbonate, silicon dioxide, aluminum hydroxide, calcium hydrogen phosphate, calcium pyrophosphate, etc., but these abrasives do not have an antibacterial function, and cannot inhibit the adhesion of dental plaque and prevent periodontitis or dental caries during brushing. With the improvement of people's living standards and health awareness, toothpaste with good antibacterial function is welcomed by the public. At present, in the product design of antibacterial toothpaste, it mainly depends on the addition of antibacterial ingredients to achieve the antibacterial function of the toothpaste, but these methods have the disadvantages of poor long-term biocompatibility, safety and antibacterial stability.

### SUMMARY

The objective of the present application is to provide an antibacterial toothpaste to achieve the dual effects of cleaning teeth and resisting bacteria. The specific technical solutions are as follows: The present application provides an antibacterial toothpaste, comprising a charged antibacterial toothpaste abrasive, wherein the charged antibacterial toothpaste abrasive comprises a ferroelectric ceramic selected from at least one or a compound or a composition of one or more of potassium sodium niobate K_{0.5}Na_{0.5}NbO₃, barium titanate BaTiO₃, lithium niobate LiNbO₃, and barium strontium titanate BaₓSr₁₋ₓTiO₃, wherein 0<x<1.

The antibacterial toothpaste provided by the present application uses a charged antibacterial toothpaste abrasive comprising the ferroelectric ceramic. Due to the spontaneous polarization property of the ferroelectric ceramic and its ability to maintain good electrical stability, the residual polarization effect of the ferroelectric ceramic is utilized to realize the inhibition of bacteria and dental plaque on the surface of teeth, so that the antibacterial toothpaste can not only clean the teeth during brushing, but also play an antibacterial role to realize the functions of inhibiting bacteria, inhibiting dental plaque, and preventing or alleviating periodontal inflammation or dental caries. Moreover, the charged antibacterial toothpaste abrasive comprising the ferroelectric ceramic can improve the long-term compatibility, safety and antibacterial stability of the antibacterial toothpaste. Certainly, any product or method in the present application does not necessarily need to achieve all the foregoing advantages at the same time.

### BRIEF DESCRIPTION OF THE DRAWINGS

In order to more clearly illustrate the technical solutions in the examples of the present application or in the prior art, the drawings required for describing the examples or the prior art are briefly described below. Apparently, the drawings in the following description are only some examples of the present application. For those of ordinary skill in the art, other examples can be obtained according to these drawings.

FIG. 1 shows electron microscope images of the ferroelectric ceramics in Examples 1-5.

FIG. 2 shows X-ray diffraction patterns of the ferroelectric ceramics in Examples 1-5.

FIG. 3 shows the antibacterial rates of the antibacterial toothpastes in Examples 1 and 5 under cold-heat cycles.

FIG. 4 shows the antibacterial rates of the antibacterial toothpaste in Example 3 under variable temperature condition and constant temperature condition.

FIG. 5 shows the plaque removal rates of the toothpastes in Example 3, Example 6, and Comparative Examples 1-2.

FIG. 6 shows the antibacterial effects of different amounts of BaTiO₃ fibers under variable temperature condition and constant temperature condition.

FIG. 7 shows the comparison result of the ability of the electroactive toothpaste (containing Ba_{0.7}Sr_{0.3}TiO₃) and a commercially available fluorine-containing toothpaste (fluorine content: 0.1%) to inhibit bacterial proliferation.

FIG. 8 shows the comparison result of the cleaning effects of the electroactive toothpaste (containing Ba_{0.7}Sr_{0.3}TiO₃) and a commercially available fluorine-containing toothpaste (fluorine content: 0.1%).

FIG. 9 shows the comparison result of the inhibiting and killing effect of the electroactive toothpaste (containing Ba_{0.7}Sr_{0.3}TiO₃) and a commercially available fluorine-containing toothpaste (fluorine content: 0.1%) on bacteria.

### DETAILED DESCRIPTION

The technical solutions of the examples of the present application will be clearly and completely described below with reference to the drawings of the examples of the present application. It should be understood that the described examples are merely part of the examples of the present application, rather than all of them. Based on the examples in the present application, all other examples obtained by those of ordinary skill in the art shall fall within the scope of protection of the present application.

The present application provides an antibacterial toothpaste, comprising a charged antibacterial toothpaste abrasive, wherein the charged antibacterial toothpaste abrasive comprises a ferroelectric ceramic selected from at least one or a compound or a composition of one or more of potassium sodium niobate K_{0.5}Na_{0.5}NbO₃, barium titanate BaTiO₃, lithium niobate LiNbO₃, and barium strontium titanate BaₓSr₁₋ₓTiO₃, wherein 0<x<1.

The antibacterial toothpaste provided by the present application uses a charged antibacterial toothpaste abrasive comprising the ferroelectric ceramic. Due to the spontaneous polarization property of the ferroelectric ceramic and its ability to maintain good electrical stability, the residual polarization effect of the ferroelectric ceramic is utilized to realize the inhibition of bacteria and dental plaque on the surface of teeth, so that the antibacterial toothpaste can not only clean the teeth during brushing, but also play an antibacterial role through the continuous and stable charging property of the charged antibacterial toothpaste abrasive, and the antibacterial performance is good in a lasting manner, thereby realizing the functions of inhibiting the bacterial activity on the surface of the teeth, inhibiting the adhesion of dental plaque, and preventing or alleviating periodontal inflammation or dental caries.

In some embodiments of the present application, the ferroelectric ceramic is subjected to corona polarization treatment in advance; the corona polarization treatment parameters comprise a polarization voltage of 1-30 kV, a polarization distance of 1-50 mm, and a polarization time of 1-60 min.

In some embodiments of the present application, the ferroelectric ceramic comprises ferroelectric ceramic particles or ferroelectric ceramic fibers.

In some embodiments of the present application, the ferroelectric ceramic particles have a particle size of 1-3 μm; the ferroelectric ceramic fibers have a length-to-diameter ratio of 1-6, a length of 1-6 μm, and a diameter of 200-400 nm.

In the present application, ferroelectric ceramic particles with a particle size of 1-3 μm or ferroelectric ceramic fibers with a length-to-diameter ratio of 1-6 are used, so that the charged antibacterial toothpaste abrasive can have a uniform structure and stable electrical properties, thereby exerting a better antibacterial effect and reducing the degree of wear on teeth when teeth are cleaned. In the present application, when the ferroelectric ceramic particles with a particle size of 1-3 μm are used, fine particles are not easy to cause foreign body sensation, and flocculation and re-agglomeration caused by too small particles (e.g., less than 1 μm) can be avoided.

In some embodiments of the present application, the charged antibacterial toothpaste abrasive has a Mohs hardness of 2-4.

In some embodiments of the present application, by weight percent based on the weight of the antibacterial toothpaste, the antibacterial toothpaste comprises 10-50 wt % of the charged antibacterial toothpaste abrasive: preferably, the antibacterial toothpaste comprises 20-40 wt % of the charged antibacterial toothpaste abrasive.

In the present application, when the weight percentage of the charged antibacterial toothpaste abrasive in the antibacterial toothpaste is 10-50 wt %, the antibacterial performance of the antibacterial toothpaste is good, and the antibacterial rate of the antibacterial toothpaste is in the range of 15-99.99%; when the weight percentage of the charged antibacterial toothpaste abrasive in the antibacterial toothpaste is 20-40 wt %, the antibacterial performance of the antibacterial toothpaste is better, and the antibacterial rate of the antibacterial toothpaste is in the range of 30-99.99%.

In some embodiments of the present application, the antibacterial toothpaste further comprises a conventional toothpaste abrasive selected from at least one of calcium carbonate, silicon dioxide, aluminum hydroxide, calcium hydrogen phosphate, and calcium pyrophosphate: by weight percent based on the weight of the antibacterial toothpaste, the total weight of the charged antibacterial toothpaste abrasive and the conventional toothpaste abrasive is 10-50 wt %, and the weight ratio of the charged antibacterial toothpaste abrasive to the conventional toothpaste abrasive is 1:(0-1); preferably, the total weight of the charged antibacterial toothpaste abrasive and the conventional toothpaste abrasive is 20-40 wt %.

In some embodiments of the present application, when the weight percentage of the charged antibacterial toothpaste abrasive in the antibacterial toothpaste is 20-40 wt % and the conventional toothpaste abrasive is not contained, the antibacterial rate of the antibacterial toothpaste is in the range of 40-99.99%.

In some embodiments of the present application, the antibacterial toothpaste has an antibacterial rate of 15-99.99%; preferably, the antibacterial toothpaste has an antibacterial rate of 30-99.99%; more preferably, the antibacterial toothpaste has an antibacterial rate of 40-99.99%.

The preparation method for the ferroelectric ceramic particles is not particularly limited in the present application, as long as the objective of the present application can be achieved. Exemplarily, in the present application, the preparation method for the ferroelectric ceramic particles comprises:
(1) weighing out a ferroelectric ceramic raw material, mixing well, and levigating to obtain a mixture;
(2) calcining the mixture at a high temperature to generate the ferroelectric ceramic through a solid-phase reaction, and grinding to obtain ferroelectric ceramic powder;
(3) subjecting the ferroelectric ceramic powder to corona polarization treatment to obtain the ferroelectric ceramic particles, wherein the corona polarization treatment parameters comprise a polarization voltage of 1-30 kV, a polarization distance of 1-50 mm, and a polarization time of 1-60 min.

In step (1), the ferroelectric ceramic raw material may be an oxide, a carbonate, a nitrate, or the like of a corresponding element, which is not particularly limited in the present application, as long as the objective of the present application can be achieved. Before the ferroelectric ceramic raw material is weighed out, the ferroelectric ceramic raw material is pretreated to remove impurities and remove moisture. In the present application, the mixing well and levigating method is not particularly limited, as long as the objective of the present application can be achieved. For example, dry grinding or wet grinding can be used, and stirring ball milling or jet milling can also be used.

In step (2), calcination is performed at a high temperature for a solid-phase reaction, so that the prepared ferroelectric ceramic can be densified and then ground to obtain ferroelectric ceramic powder with uniform components and an appropriate particle size.

In step (3), after the corona polarization treatment, the ferroelectric ceramic particles have a certain polarization charge on the surface thereof and have a continuous and stable charging performance. The preparation method for the ferroelectric ceramic particles of the present application is simple, efficient, low-cost, and strong in controllability, and can be used for industrial production.

The preparation method for the ferroelectric ceramic fibers is not particularly limited in the present application, as long as the objective of the present application can be achieved. Exemplarily, the ferroelectric ceramic fibers of the present application can be prepared by an electrospinning method.

The antibacterial toothpaste of the present application uses the charged antibacterial toothpaste abrasive of the present application, and other components may include a wetting agent, a binder, a foaming agent, a preservative, a flavoring agent, a sweetening agent, and water. The present application is not particularly limited as long as the objective of the present application can be achieved.

The preparation method for the antibacterial toothpaste is not particularly limited in the present application, as long as the objective of the present application can be achieved.

The embodiments of the present application will be described in more detail below with reference to the examples and comparative examples.

### Test Method and Equipment:

Particle size test: The particle size distribution of the ferroelectric ceramic particles was measured under an electron microscope and statistically analyzed.
Hardness test: A sharp point on the tested sample of the charged antibacterial toothpaste abrasive was selected. The hardness test was conducted in ascending order on the flat surface of a plane mineral hardness tester with known hardness. The tester surface was observed to see whether any scratches appeared. The surface was gently wiped to ensure that the powder from the tested sample remained on the tester, so as to avoid misjudgment. If scratches appeared on the tester surface, it indicated that the sample hardness was greater than that of the tester. Testers of increasing hardness were used sequentially until the hardness fell between two levels or corresponded to a specific tester level, thereby determining the Mohs hardness of the tested charged antibacterial toothpaste abrasive.
Cleaning performance test: (1) Bovine teeth were selected as stain carriers. After preliminary sandblasting and cleaning, they were sequentially immersed in an albumin solution, a mixture of tea and coffee, and a ferric citrate solution for 30 min. This process was repeated until the stains were firmly attached to the surface of the bovine teeth for later use. The stained bovine teeth were placed in the sample slot of a brushing machine, ensuring that the stain surface was level with the surface of the sample slot. The brush head and force arm were adjusted to conform to the stain surface. The toothpaste dispersion was poured into the sample slot, and a specified load was applied. A simulated brushing test was conducted according to predetermined conditions. The stain cleaning capability was measured by image changes. (2) Bovine teeth and oral dental plaque bacteria were co-cultured in BHI liquid medium (brain heart infusion medium) for 12 h. The medium was pipetted out using a pipette, and the teeth were gently washed once with sterile normal saline to remove the suspended bacteria. The bovine teeth were placed in the sample slot of a brushing machine, ensuring that the stain surface was level with the surface of the sample slot. The brush head and force arm were adjusted to conform to the stain surface. The toothpaste dispersion was poured into the sample slot, and a specified load was applied. A simulated brushing test was conducted according to predetermined conditions. An appropriate amount of dye solution was dropped onto the surface of the bovine teeth and incubated in the dark at room temperature for 15 min. The teeth were carefully rinsed with phosphate buffered saline (PBS buffer) to remove excess dye. A laser confocal microscope (CLSM) was used to observe and capture images, obtaining the green fluorescence intensity. The plaque cleaning rate was obtained by comparing with the control group without toothpaste treatment according to (1−green fluorescence intensity of toothpaste group/green fluorescence intensity of control group)×100%.
Antibacterial performance test: The toothpaste was taken and co-incubated with 300 μL (10⁴ CFU/mL) of Streptococcus mutans through 25 cold-heat cycles (20-45° C. variable) or at a constant temperature (37° C.), and the number of bacteria in the bacteria liquid was counted: the bacterial activity rate of the toothpaste was calculated according to bacterial activity rate=(number of bacteria before culture−number of bacteria after culture)/number of bacteria before culture×100%; the antibacterial rate was calculated according to antibacterial rate=1−bacterial activity rate.

### Example 1

### Preparation of Charged Antibacterial Toothpaste Abrasive

BaTiO₃ particles were subjected to corona polarization treatment to obtain polarized BaTiO₃ particles, which were used as the charged antibacterial toothpaste abrasive. The corona polarization treatment parameters comprise a polarization voltage of 20 kV, a polarization distance of 25 mm, and a polarization time of 30 min.

### Preparation of Toothpaste

The toothpaste comprises, by weight percent based on the weight of the toothpaste, a charged antibacterial toothpaste abrasive: BaTiO₃ particles 20 wt %: a wetting agent: sorbitol 61 wt %; a binder: hydroxymethyl cellulose sodium 1 wt %; a foaming agent: sodium lauryl sulfate 2 wt %; a preservative: parabens 3 wt %; a flavoring agent: essence 1 wt %; a sweetening agent: saccharin 0.3%; and the balance being deionized water.

The above components were weighed out according to their weight percentages. The wetting agent was placed into a paste-making machine. The preservative, flavoring agent, and sweetening agent were dissolved in water, stirred in a liquid tank until uniform, and then added to the paste-making machine. The mixture was stirred for 10 min until evenly mixed. The toothpaste abrasive, binder, and foaming agent were mixed evenly and then added to the paste-making machine. The mixture was stirred for 15 min until evenly mixed. The mixture was then degassed to obtain the toothpaste.

### Example 2

### Preparation of Charged Antibacterial Toothpaste Abrasive

Ba_{0.5}Sr_{0.2} TiO₃ particles were subjected to corona polarization treatment to obtain polarized Ba_{0.5}Sr_{0.2} TiO₃ particles, which were used as the charged antibacterial toothpaste abrasive. The corona polarization treatment parameters comprise a polarization voltage of 5 kV, a polarization distance of 3 mm, and a polarization time of 50 min.

### Preparation of Toothpaste

The toothpaste comprises, by weight percent based on the weight of the toothpaste, a charged antibacterial toothpaste abrasive: Ba_{0.5}Sr_{0.2}TiO₃ particles 40 wt %; a wetting agent: sorbitol 50 wt %; a binder: hydroxymethyl cellulose sodium 1 wt %; a foaming agent: sodium lauryl sulfate 0.5 wt %; a preservative: parabens 2 wt %; a flavoring agent: essence 0.8 wt %; a sweetening agent: saccharin 0.3%; and the balance being deionized water.

The toothpaste was prepared by the same procedure as in Example 1.

### Example 3

### Preparation of Charged Antibacterial Toothpaste Abrasive

Ba_{0.7}Sr_{0.3}TiO₃ particles were subjected to corona polarization treatment to obtain polarized Ba_{0.7}Sr_{0.3}TiO₃ particles, which were used as the charged antibacterial toothpaste abrasive. The corona polarization treatment parameters comprise a polarization voltage of 25 kV, a polarization distance of 35 mm, and a polarization time of 30 min.

### Preparation of Toothpaste

The procedure was the same as that in Example 2 except that the type of the charged antibacterial toothpaste abrasive was replaced by Ba_{0.7}Sr_{0.3}TiO₃ particles.

### Example 4

### Preparation of Charged Antibacterial Toothpaste Abrasive

Ba_{0.6}Sr_{0.4}TiO₃ particles were subjected to corona polarization treatment to obtain polarized Ba_{0.6}Sr_{0.4}TiO₃ particles, which were used as the charged antibacterial toothpaste abrasive. The corona polarization treatment parameters comprise a polarization voltage of 30 kV, a polarization distance of 18 mm, and a polarization time of 5 min.

### Preparation of Toothpaste

The procedure was the same as that in Example 2 except that the type of the charged antibacterial toothpaste abrasive was replaced by Ba_{0.6}Sr_{0.4}TiO₃ particles.

### Example 5

### Preparation of Charged Antibacterial Toothpaste Abrasive

(1) 9 mL of glacial acetic acid was mixed with 1.340 g of acetylacetone and stirred for 5 min under plastic wrap sealing: 1.703 g of barium acetate was added, and the mixture was stirred for 1 h until completely dissolved: 2.266 g of tetrabutyl titanate was added, and the mixture was stirred for 15 min: 0.45 g of polyvinylpyrrolidone was added, and the mixture was stirred for 2-3 h until completely dissolved to obtain the electrospinning solution.
(2) The electrospinning solution was subjected to electrospinning treatment, dried at 70° C. overnight for 12 h, warmed in a muffle furnace at a ramp rate of 5° C./min to 500° C., calcined for 2 h, further warmed to 750° C., calcined for 2 h, and naturally cooled to obtain BaTiO₃ fibers. The parameters for the electrospinning treatment included a voltage: a positive voltage of 7.5 kV and a negative voltage of 7.5 kV: a bolus speed: 0.035 mm/min, syringe model 22 g: a tip-to-roller distance of 15 cm; a roller rotational speed of 800 rpm.

BaTiO₃ fibers were subjected to corona polarization treatment to obtain polarized BaTiO₃ fibers, which were used as the charged antibacterial toothpaste abrasive. The corona polarization treatment parameters were the same as those in Example 1.

### Preparation of Toothpaste

The procedure was the same as that in Example 1 except that the type of the charged antibacterial toothpaste abrasive was replaced by BaTiO₃ fibers.

### Example 6

### Preparation of Charged Antibacterial Toothpaste Abrasive

Ba_{0.7}Sr_{0.3}TiO₃ particles were subjected to corona polarization treatment to obtain polarized Ba_{0.7}Sr_{0.3}TiO₃ particles, which were used as the charged antibacterial toothpaste abrasive. The corona polarization treatment parameters comprise a polarization voltage of 25 kV, a polarization distance of 35 mm, and a polarization time of 30 min.

### Preparation of Toothpaste

The toothpaste comprises, by weight percent based on the weight of the toothpaste, a charged antibacterial toothpaste abrasive: Ba_{0.7}Sr_{0.3}TiO₃ particles 20 wt %; a conventional toothpaste abrasive: SiO₂ 20 wt %; a wetting agent: sorbitol 50 wt %; a binder: hydroxymethyl cellulose sodium 1 wt %; a foaming agent: sodium lauryl sulfate 0.5 wt %; a preservative: parabens 2 wt %; a flavoring agent: essence 0.8 wt %; a sweetening agent: saccharin 0.3%; and the balance being deionized water. The toothpaste was prepared by the same procedure as in Example 1.

### Example 7

### Preparation of Charged Antibacterial Toothpaste Abrasive

Ba_{0.7}Sr_{0.3}TiO₃ particles were subjected to corona polarization treatment to obtain polarized Ba_{0.7}Sr_{0.3}TiO₃ particles, which were used as the charged antibacterial toothpaste abrasive. The corona polarization treatment parameters were the same as those in Example 3.

### Preparation of Toothpaste

The toothpaste comprises, by weight percent based on the weight of the toothpaste, a charged antibacterial toothpaste abrasive: Ba_{0.7}Sr_{0.3}TiO₃ particles 10 wt %; a wetting agent: sorbitol 65 wt %; a binder: hydroxymethyl cellulose sodium 1 wt %; a foaming agent: sodium lauryl sulfate 2 wt %; a preservative: parabens 3 wt %; a flavoring agent: essence 1 wt %; a sweetening agent: saccharin 0.3%; and the balance being deionized water.

The toothpaste was prepared by the same procedure as in Example 1.

### Comparative Example 1

### Preparation of Toothpaste

The procedure was the same as that in Example 1 except that the type of the toothpaste abrasive was replaced by SiO₂.

### Comparative Example 2

### Preparation of Toothpaste

The procedure was the same as that in Example 1 except that the type of the toothpaste abrasive was replaced by CaCO₃.

The hardness of each charged antibacterial toothpaste abrasive after polarization in Examples 1-4 was tested, and the Mohs hardness was measured to be 3 for all samples.

Table 1 shows the performance parameters of the toothpaste prepared in each example and comparative example.

TABLE 1

| | | | Particle size | | | |
|---|---|---|---|---|---|---|
| | | Total | (μm) of charged | | | |
| | | content of | antibacterial | Cleaning | Antibacterial rate | |
| | | toothpaste | toothpaste abrasive | rate of | of toothpaste (%) | |
| | Type of toothpaste | abrasive in | particle/fiber length- | toothpaste | Constant | Variable |
| / | abrasive | toothpaste | to-diameter ratio | (%) | temperature | temperature |
| Example 1 | BaTiO₃ particle | 20 wt % | 2 | / | / | 64.7 |
| Example 3 | Ba_{0.7}Sr_{0.3} TiO₃ particle | 40 wt % | 3 | 99.7 ± 0.2 | 41.3 | 99.5 |
| Example 5 | BaTiO₃ fiber | 20 wt % | 5 | / | 47.11 | 60.72 |
| Example 6 | Ba_{0.7}Sr_{0.3}TiO₃ particle | 40 wt % | 3 | 96.9 ± 1.2 | 34.7 | 67.5 |
| | and SiO₂ in a weight | | | | | |
| | ratio of 1:1 | | | | | |
| Example 7 | Ba_{0.7}Sr_{0.3}TiO₃ particle | 10 wt % | 3 | / | 15.6 | 33 |
| Comparative | SiO₂ | 40 wt % | / | 91.2 ± 6.3 | / | / |
| Example 1 | | | | | | |
| Comparative | CaCO₃ | 40 wt % | / | 87.2 ± 7.6 | / | / |
| Example 2 | | | | | | |

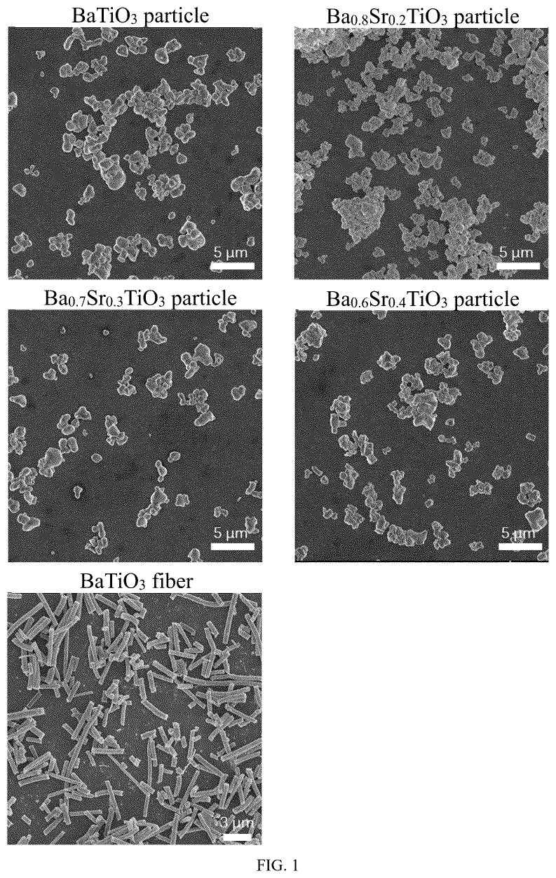

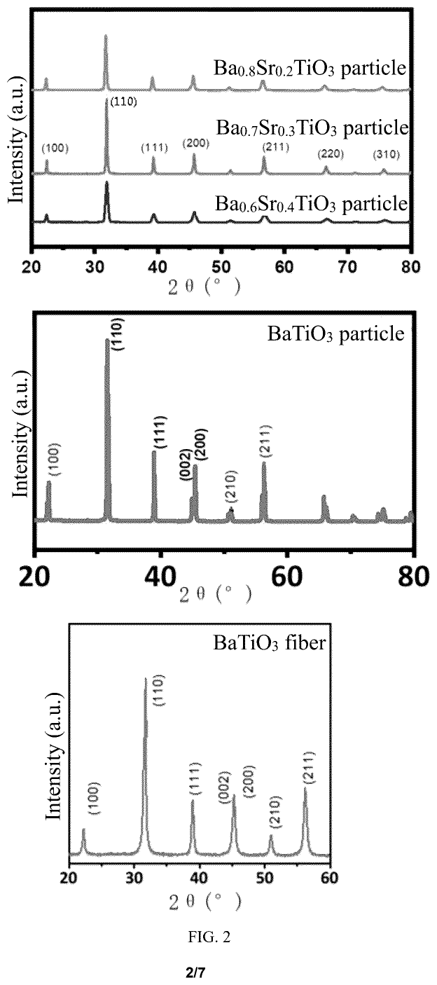

The electron microscope images of the ferroelectric ceramics in Examples 1-5 are shown in FIG. 1, and the X-ray diffraction (XRD) patterns of the ferroelectric ceramics in Examples 1-5 are shown in FIG. 2. These results indicate that the ferroelectric ceramic particles of the present application exhibit a tetragonal crystal structure, possess ferroelectricity and are capable of spontaneous polarization.

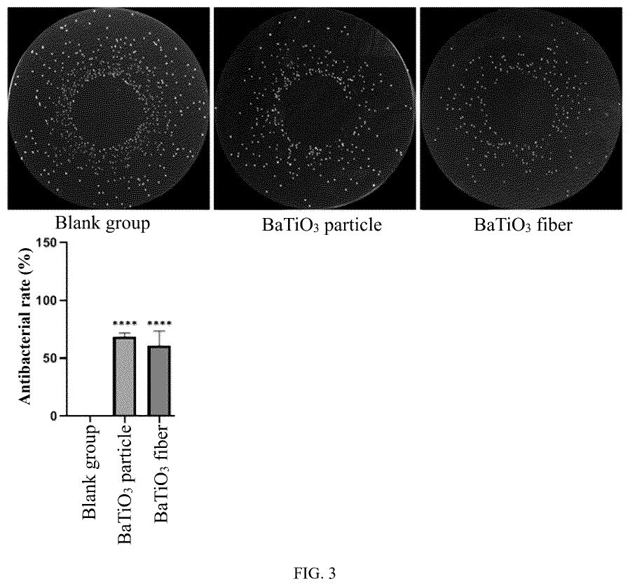

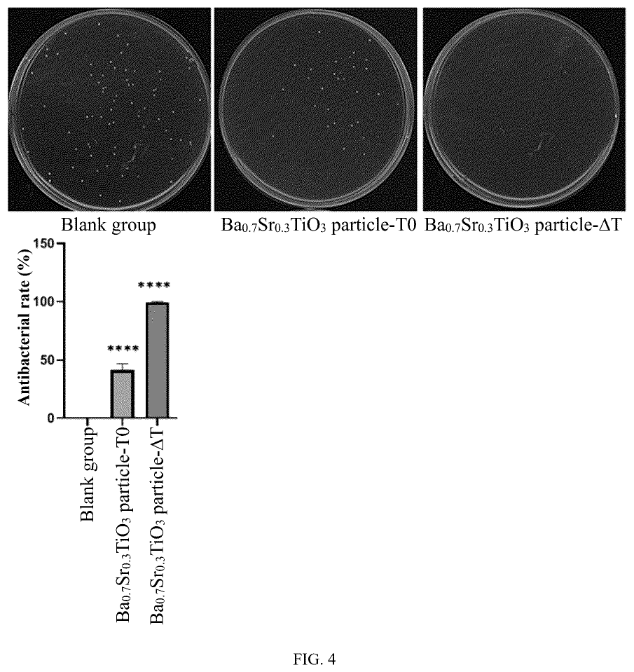

The antibacterial rates of the antibacterial toothpastes in Examples 1 and 5 under cold-heat cycles are shown in FIG. 3. The antibacterial rates of the antibacterial toothpaste in Example 3 under both variable temperature condition (ΔT) and constant temperature condition (T0) are shown in FIG. 4. The plaque removal rates of the toothpastes in Examples 3 and 6, as well as in Comparative Examples 1 and 2, are shown in FIG. 5.

According to FIG. 3, FIG. 4, and Table 1, in the antibacterial performance tests, the blank group without toothpaste exhibited significantly higher bacterial counts. The antibacterial toothpastes containing the charged antibacterial toothpaste abrasives of the present application significantly improved the antibacterial rates (****P<0.0001 vs. blank group), demonstrating strong antibacterial performance. According to the results of Examples 3 and 6 in Table 1, the charged antibacterial toothpaste abrasives of the present application could also be combined with conventional toothpaste abrasives, and the resulting antibacterial toothpaste still exhibited good antibacterial performance.

As shown in Table 1 and FIG. 5, the plaque removal rates of the antibacterial toothpastes of the present application were comparable to those of the toothpastes in Comparative Examples 1 and 2 (statistical result: P=0.127>0.05; no significant difference between groups).

In conclusion, the antibacterial toothpastes of the present application demonstrated dual effects of resisting bacteria and cleaning teeth.

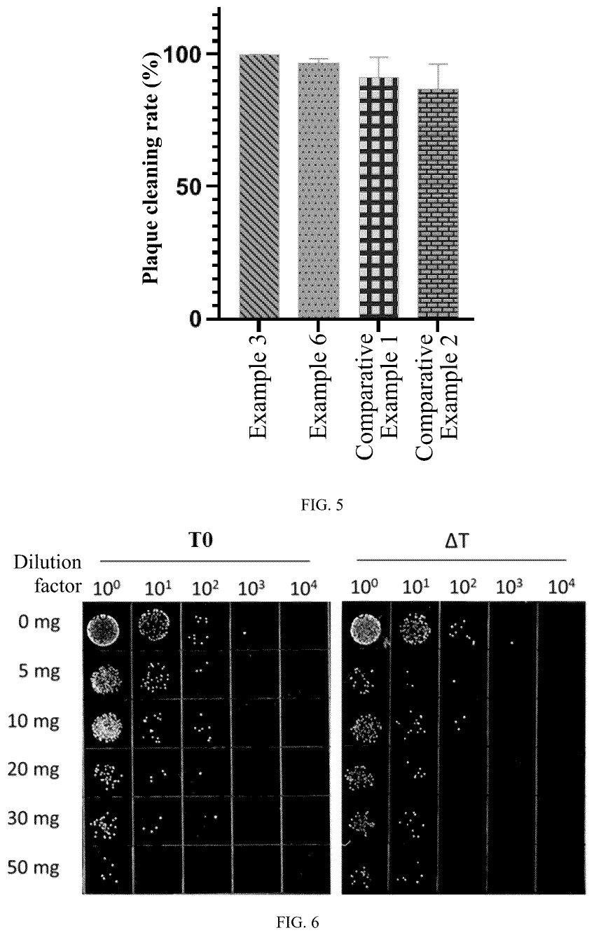

In the present application, polarized BaTiO₃ fibers in Example 5, with amounts of 0 mg (blank control), 5 mg, 10 mg, 20 mg, 30 mg, and 50 mg, were used to test the antibacterial performance of the ferroelectric ceramic (100) under constant temperature conditions (T0) and cold-heat cycle conditions (ΔT), which were diluted 101, 102, 103, and 104 times with PBS buffer, respectively, to obtain the antibacterial effects of the BaTiO₃ fibers as shown in FIG. 6. From FIG. 6, the antibacterial effects of the BaTiO₃ fibers under variable temperature and constant temperature conditions were both positively correlated with the amount used. As the amount of BaTiO₃ fibers increased, the antibacterial effect showed an upward trend.

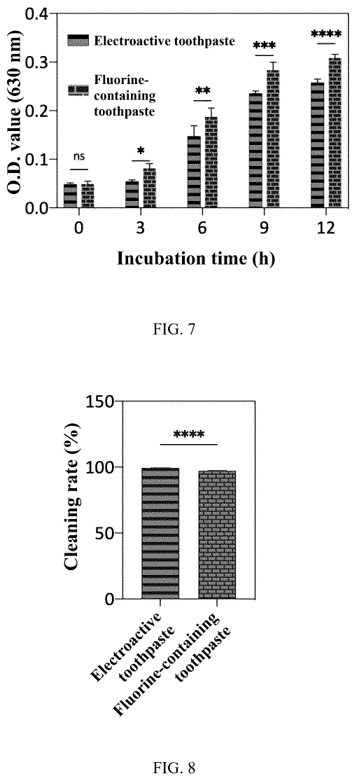

The present application further compared the ability to inhibit bacterial proliferation of the electroactive toothpaste (containing BaTiO₃ in Example 3) and commercially available fluorine-containing toothpaste (fluorine content: 0.1%), as shown in FIG. 7. In the quantitative analysis of microorganisms, OD values are commonly used to reflect the concentration or number of microorganisms. Dental caries mixed bacteria were co-cultured with the electroactive toothpaste (containing BST) and commercially available fluorine-containing toothpaste (fluorine content: 0.1%), and the OD values of the bacterial suspensions were measured at 0, 3, 6, 9, and 12 h, respectively, by a wavelength of 630 nm. A higher OD value indicated a higher total bacterial count. The present application also compared the cleaning effect of the electroactive toothpaste (containing Ba_{0.7}Sr_{0.3}TiO₃ in Example 3) and commercially available fluorine-containing toothpaste (fluorine content: 0.1%), as shown in FIG. 8. The dental caries mixed bacteria were co-cultured with bovine teeth for 24 h to allow the bacteria to colonize on the tooth surface. The electroactive toothpaste (containing Ba_{0.7}Sr0.3TiO3) and commercially available fluorine-containing toothpaste (fluorine content: 0.1%) were then used to clean the bovine teeth using the Bass brushing method. After cleaning, a bacterial viable/dead staining solution was added dropwise for incubation in the dark for 15 min, and then the fluorescence expression level of the viable/dead bacteria was detected by laser confocal microscopy. The cleaning rate was calculated as: (red fluorescence+green fluorescence)/blank control group*100% (red fluorescence represents dead bacteria, and green fluorescence represents live bacteria).

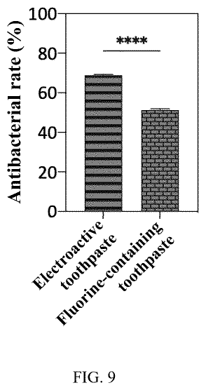

The present application also compared the inhibiting and killing effects of the electroactive toothpaste (containing Ba_{0.7}Sr_{0.3}TiO₃ in Example 3) and commercially available fluorine-containing toothpaste (fluorine content: 0.1%) on bacteria, as shown in FIG. 9. The dental caries mixed bacteria were co-cultured with the electroactive toothpaste (containing BST) and commercially available fluorine-containing toothpaste (fluorine content: 0.1%) for 24 h. The bacterial suspension was then pipetted out, centrifuged, and precipitated. A bacterial viable/dead staining solution was added dropwise for incubation in the dark for 15 min, and the mixture was centrifuged and precipitated, washed with PBS, and centrifuged and precipitated. The fluorescence expression level of the viable/dead bacteria was detected by laser confocal microscopy. The antibacterial rate was calculated as: red fluorescence/(red fluorescence+green fluorescence)*100% (red fluorescence represents dead bacteria, and green fluorescence represents live bacteria).

In conclusion, the antibacterial toothpaste provided by the present application comprises a charged antibacterial toothpaste abrasive, wherein the charged antibacterial toothpaste abrasive comprises a ferroelectric ceramic selected from at least one or a compound or a composition of one or more of potassium sodium niobate K_{0.5}Na_{0.5}NbO₃, barium titanate BaTiO₃, lithium niobate LiNbO₃, and barium strontium titanate BaₓSr₁₋ₓTiO₃, wherein 0<x<1. In the antibacterial toothpastes of the present application, the charged antibacterial toothpaste abrasive can maintain good electrical stability, and the antibacterial toothpaste containing the charged antibacterial toothpaste abrasive can play a long-lasting antibacterial role by utilizing the polarization effect, and has good antibacterial performance, so as to achieve the dual effects of cleaning teeth and resisting bacteria. It should be noted that, the term “comprise”, “include”, or any other variations thereof herein is intended to cover non-exclusive inclusion, such that a process, method, article, or apparatus that comprises a list of elements does not include only those elements, but may also include other elements not expressly listed, or may include elements inherent to such process, method, article, or apparatus. Without further limitation, an element defined by the phrase “comprising a . . . ” does not exclude the presence of additional identical elements in a process, method, article, or apparatus that comprises the element.

The various examples described in this specification are presented in a related manner, and reference may be made among the examples for similar or identical parts. Each example primarily illustrates the differences from other examples.

The foregoing description is merely preferred examples of the present application and is not intended to limit the scope of protection of the present application. Any modifications, equivalent substitutions, or improvements made within the spirit and principles of the present application shall fall within the scope of protection of the present application.

1. An antibacterial toothpaste, comprising a charged antibacterial toothpaste abrasive, wherein the charged antibacterial toothpaste abrasive comprises a ferroelectric ceramic, and the ferroelectric ceramic is barium strontium titanate BaₓSr₁₋ₓTiO₃, wherein 0<x<1;
the ferroelectric ceramic comprises ferroelectric ceramic particles or ferroelectric ceramic fibers;
the ferroelectric ceramic particles have a particle size of 1-3 μm; the ferroelectric ceramic fibers have a length-to-diameter ratio of 1-6, a length of 1-6 μm, and a diameter of 200-400 nm;
by weight percent based on a weight of the antibacterial toothpaste, a total weight of the charged antibacterial toothpaste abrasive and a conventional toothpaste abrasive is 40-50 wt %, and a weight ratio of the charged antibacterial toothpaste abrasive to the conventional toothpaste abrasive is 1:(0-1).

2. The antibacterial toothpaste according to claim 1, wherein the ferroelectric ceramic is previously subjected to corona polarization treatment.

3. The antibacterial toothpaste according to claim 2, wherein the corona polarization treatment parameters comprise a polarization voltage of 1-30 kV, a polarization distance of 1-50 mm, and a polarization time of 1-60 min.

4. The antibacterial toothpaste according to claim 1, wherein the charged antibacterial toothpaste abrasive has a Mohs hardness of 2-4.

5. The antibacterial toothpaste according to claim 1, wherein the conventional toothpaste abrasive is selected from at least one of calcium carbonate, silicon dioxide, aluminum hydroxide, calcium hydrogen phosphate, and calcium pyrophosphate.

6. The antibacterial toothpaste according to claim 1, wherein the antibacterial toothpaste has an antibacterial rate of 15-99.99%.

7. The antibacterial toothpaste according to claim 6, wherein the antibacterial toothpaste has an antibacterial rate of 30-99.99%.

8. The antibacterial toothpaste according to claim 7, wherein the antibacterial toothpaste has an antibacterial rate of 40-99.99%.
